(19) United States
(12) Patent Application Publication
(10) Pub. No.: US 2004/0121031 A1
(43) Pub. Date: Jun. 24, 2004

(54) Novel topical skin care and nutraceutical applications of Glabridin or extracts containing a defined amount (4-90%) of Glabridin

(76) Inventors: Muhammed Majeed, Piscataway, NJ (US); Kalkunte Seshadri Satyan, Bangalore (IN); Kanhangad Gangadharan Geetha, Bangalore (IN); Subbalakshmi Prakash, Piscataway, NJ (US)

Correspondence Address: SABINSA CORPORATION, 121 ETHEL ROAD WEST, UNIT 6, PISCATAWAY, NJ, 08854, US

(21) Appl. No.: 10/604,710

(22) Filed: Aug. 12, 2003

Related U.S. Application Data
(63) Continuation-in-part of application No. 10/065,995, filed on Dec. 9, 2002.

(51) Int. Cl. A61K 35/78

(52) U.S. Cl. 424/757

(57) ABSTRACT

This application is a continuation-in-part of pending U.S. patent application Ser. No. 10/065,995 by the authors, filed on Dec. 9, 2002, for a Commercial Process for Isolation and Purification of Glabridin with High Tyrosinase Inhibitory Activity and its Cosmetic Compositions and Methods Of Use. The current invention discloses the use of Glabridin containing Licorice extract (4-90% Glabridin) as metalloprotease and hyaluronidase inhibiting component in cosmetic topical or oral formulations. These extracts and particularly Glabridin, are useful in anti-wrinkle and anti-aging products, providing elasticity, firmness, tone and texture to the skin, ameliorating fine lines and crows feet in under eye preparations, and prevent skin and hair damage due to UV rays, inflammation and itch, diaper rashes in baby products, as massage or toning oils or emulsion for babies.

### CROSS REFERENCE TO RELATED APPLICATIONS

This application is a continuation-in-part of pending U.S. patent application Ser. No. 10/065,995 filed on Dec. 9, 2002, for a Commercial Process for Isolation and Purification of Glabridin with High Tyrosinase Inhibitory Activity and its Cosmetic Compositions and Methods Of Use, the disclosure of which is hereby incorporated by reference.

### BACKGROUND OF INVENTION

1. Field of Invention

The present invention relates to additional cosmetic, particularly skin care, applications and compositions with glabridin. More particularly, in addition to the invention described in the parent patent application, the current invention discloses the uses of Glabridin as such, or in the form of licorice extract containing a 4-90% glabridin as metalloprotease and hyaluronidase inhibiting component in formulations for topical or oral use. Glabridin is useful in anti-wrinkle and anti-aging products, providing elasticity, firmness, tone and texture to the skin, ameliorating fine lines and crows feet in under eye preparations, and prevent skin and hair damage due to UV rays, inflammation and itch, diaper rashes in baby products, as massage or toning oils or emulsion for babies.

Orally, the said extract could be used in maintaining and alleviating conditions of arthritis, joint immobility, osteoarthritis and conditions manifested due to increased activity of elastase, collagenase and hyaluronidase enzymes.

2. Description of Prior Art

Isoflavones are a larger and distinctive subclass of flavonoids. These compounds possess a 3-phenyl chromane skeleton that is biogenetically derived by rearrangement of the flavonoid 2-phenyl chromane system (1,2 diaryl rearrangement). Isoflavonoids are almost entirely distributed to the subfamily Papilionaceae of Leguminoseae family. Several flavonoids are potent inhibitors of lipoxgenase or cyclooxygenase or both. These properties explain their antiinflammatory and antiallergenic activity. Glabridin an isoflavan found in licorice extracts is reported to have anti-inflammatory, antioxidant and tyrosinase inhibitory properties (Yokota, T. et al. Pigment Cell Res. 11(6):355,361, 1998; Vaya, J. Free Rad. Biol. Med. 23(2):302-313, 1997). Methods to isolate isoflavans and other tyrosinase inhibitors have been described in literature (Mitscher, L. et al. J. Nat. Prod. 43(2):259-269, 1980; Shirota, S. et al. Biol. Pharm. Bull. 17(2):266-269, 1994; Saitoh, T. et al. Chem. Pharm. Bull. 24(4):742-755) UV induced oxidative stress leading to unbridled production of free radicals is documented to augment the activity of enzymes such as elastase, collagenase and hyaluronidase. This causes premature degradation and digestion of the key structural components elastin and collagen of the dermis.

The long term consequence of the prolonged activation of these enzymes is what is believed to lead to premature skin aging. The formation of fine lines and wrinkles or the appearance of slacking skin, is not apparently caused by direct photochemical free radical controlled reactions, but rather by the activation at the genomic level of the skin's own enzymes as a consequence of the of immune system-mediated inflammatory responses. Thus the intervention at the stage of arresting the enzyme synthesis and activity (at the genomic and the proteomic level), is essential in controlling the progress of premature skin aging, loss of elasticity, tone, loss of suppleness, inflammation, itch or irritation. (Maes D H, Marenus K D, Textbook of Cosmetic Dermatology,Ed., Robert Baran and Howard Maibach, Pbs: Martin Dunitz Ltd., 469-485, 1998). Prior art teaches that high activity of collagenase and elastase in the Synovial fluids (SF) of patients with rheumatoid arthritis (RA), which is about 30 times higher than that found in the SF of patients with osteoarthritis (OA). These findings underline the synergic action of these enzymes in the pathogenesis of joint damage. RA patients also exhibit higher levels of glutathione reductase, which is important for the detoxification pathway of oxygen free radicals. However, compared with findings for collagenase and elastase, the increase in glutathione reductase is only three times higher than level found in the SF of OA patients. (Bazzichi L, Ciompi M L, Betti L, Rossi A, Melchiorre D, Fiorini M, Giannaccini G, Lucacchini A., Clin Exp Rheumatol. 2002 November-December;20(6):761-6. Impaired glutathione reductase activity and levels of collagenase and elastase in synovial fluid in rheumatoid arthritis).

Diaper dermatitis generally called “diaper rash” manifests itself as primary irritation dermatitis brought about by physical irritation from the diaper surface, chemical irritation from stools and urine and clinically take the form of erythema, papules, edema accompanied by itching. Proteolytic and lipolytic enzymes from faeces especially elastase causes increased vasodilation, increase in transepidermal water loss and skin pH, with higher irritation potential. Prior art suggest that the possible etiologic role of proteases in perianal, circumstomal or diaper dermatitis. (Andersen P H et al., Faecal enzymes: invivo human skin irritation. Contact Dermatitis, 1994, 30(3) 152-8)

Implantation of the embryo into the endometrium is a highly regulated event that is critical for establishment of pregnancy. Molecules involved in this process provide potential targets for post-coital contraception. Administration of MMP (Matrix Metalloprotease) inhibitors in animals during early pregnancy retards decidual development. (Rechtman M P, Zhang J, Salamonsen L A., J Reprod Fertil. 1999 September;117(1):169-77). Effect of inhibition of matrix metalloproteinases on endometrial decidualization and implantation in mated rats.) Progesterone, a orally used contraceptive, is a key suppressor of the activity of MMPs in the endometrium. In cultured explants from untreated women, progesterone abrogates the expression of procollagenase-1 and the activation of progelatinase B. It also inhibits the expression of progelatinases A and B and further decreases the activities of all of these MMPs by stimulating the production of TIMP-1 (The Journal of Clinical Endocrinology & Metabolism Vol. 85, No.12 4827-4834. Christine Galant, et al., Temporal and Spatial Association of Matrix Metalloproteinases with Focal Endometrial Breakdown and Bleeding upon Progestin-Only Contraception) All of the prior art as described in the main patent application use Licorice extract.

Surprisingly, none of the prior art describe the use of glabridin, isolated from licorice roots or extracts containing a defined amount of Glabridin for elastase inhibition or collagenase or hyaluronidase inhibition and its use as anti-wrinkle, anti-aging, anti-itch or orally in the treatment of arthritis or baby care diaper rashes or in conditions of dry skin syndrome or use in preventing photoaging. In this invention, we provide evidence that glabridin from licorice extract or licorice extract containing from 4% to 90% of glabridin inhibit MMPs and hyaluronidase enzymes in skin. We claim the use of these extracts for skin and nutraceutical applications.

### SUMMARY OF INVENTION

The present invention discloses a surprising new finding that lipophilic licorice extract containing Glabridin at various purity (4%, 40% and 80%) is an excellent elastase, collagenase and hyaluronidase inhibitor. The extract containing glabridin or glabridin alone or in combination with other cosmetic acitves can be used to fight wrinkles, fine lines, premature aging, photoaging, skin tone, itch and orally for the treatment of arthritis, osteoarthritis, inflammation, tumors and as contraceptives. Compositions containing glabridin and their use in skin care are described.

The object of the present invention is to provide cosmetic compositions having metalloprotease inhibition.

It is another object of the present invention to provide compositions that have number of benefits in connection with skin care and prevent the damage to the keratinous tissue.

The compositions of this invention may be in the form of gel, lotion, anhydrous sticks, oil based sprays, oil-in-water or water-in-oil emulsion and any other oral dosage form.

These and other objects of the present invention are achieved by a cosmetic composition comprising essentially of Glabridin or an extract containing Glabridin (4%, 40% or 80%), as elastase and collagenase inhibitor along with one or more antioxidants, sunscreens, emulsifiers, preservatives, additional anti-wrinkle agents, thickeners and fragrances.

### BRIEF DESCRIPTION OF DRAWINGS

FIG. 1: Effect of content of glabridin on IC50 of Collagenase

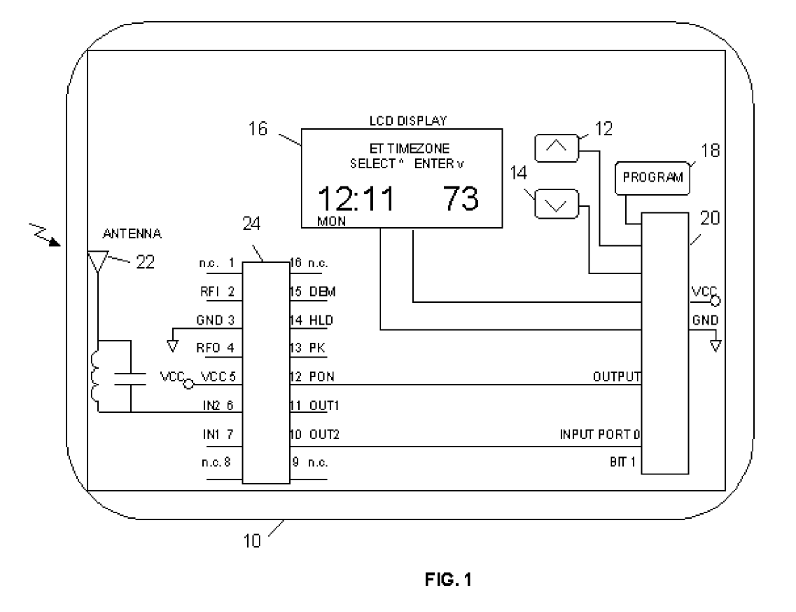

FIG. 2: Effect of content of glabridin on IC50 of Elastase

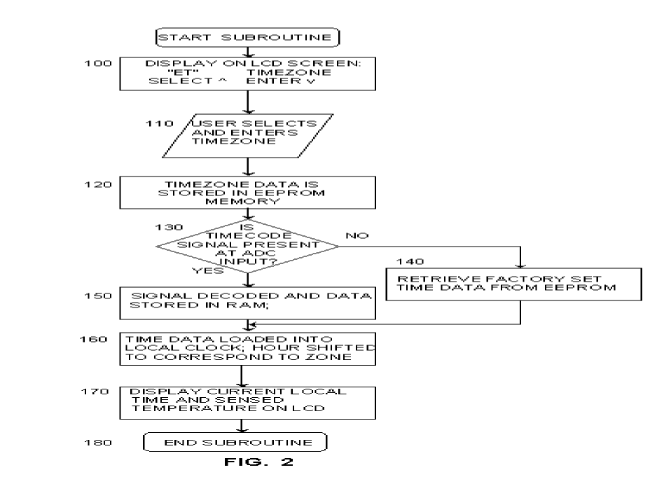

FIG. 3: Effect of content of glabridin on IC50 of Hyaluronidase

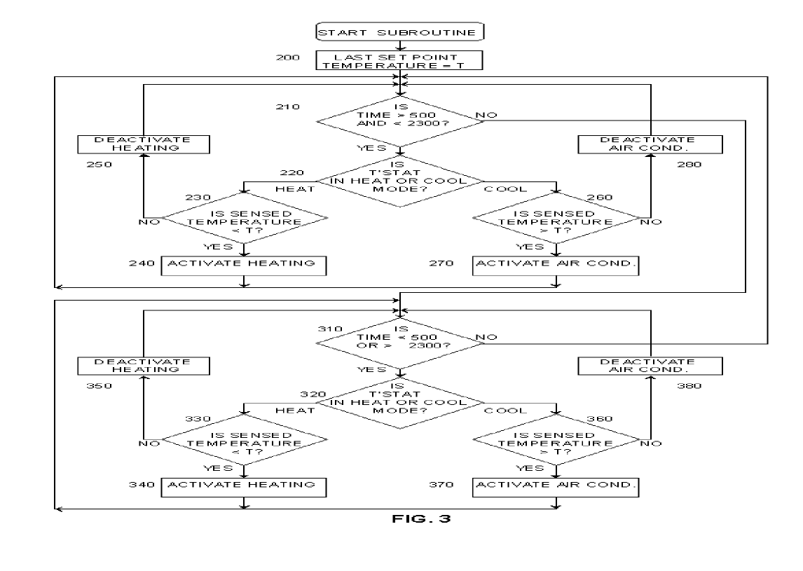

### DETAILED DESCRIPTION

The present invention, a continuation-in-part of the parent patent application, includes a skin care composition containing from about 0.001% to about 10% preferably from 0.1 to 3% most preferably from 0.1 to 0.5% by weight of a composition of purified glabridin containing 4, 20, 40 or 90% glabridin by weight and a cosmetically acceptable vehicle. The purity of glabridin in licorice extracts used in the compositions of the present invention, is selected at optimal levels to support the multiple functions of tyrosinase inhibition, metalloprotease inhibition, antioxidant effects and UV protective effects.

Glabridin of varying strengths have been studied for their inhibition of elastase, collagenase and hyaluronidase to ascertain its use as anti-aging, anti-wrinkle and anti-itch properties.

Further, in accordance with the present invention, there have been disclosed, cosmetic compositions, preferable in the form of oil-in-water emulsion. The composition contains glabridin with or without one or more of the following tyrosinase inhibitors: Tetrahydrocurcumin, Tetrahydrodemethoxycurcumin, Tetrahydrobisdemethoxycurcumin curcumin, demethoxycurcumin, bisdemethoxycurcumin, ellagic acid, soy isoflavones.

The composition may also include one or more triterpenic acids as anti-wrinkle component, antioxidants, sunscreens, emulsifiers, preservatives and thickeners.

The compositions of the present invention offer a number of benefits as anti-wrinkle, anti-aging, anti-itch , baby care diaper rashes or orally in the treatment of arthritis or in conditions of dry skin syndrome or use in preventing photoaging.

### EXAMPLE 1

### Concentration Dependent Functional Properties of Glabridin

a) Method for Anti-Collagenase Activity

The assays were done using the EnzChek collagenase assay kit. The substrate was DQ gelatin (from pig skin) (source EnzyCheck kit). DQ gelatin is a fluorescein conjugated-gelatin labeled with fluorescein. The fluorescence is quenched in the presence of inhibitor. The reduction in fluorescence intensity was measured in a microplate reader/Fluostar Optima (emission at 485 nm and excitation at 520 nm.) Procedure: Aliquots of the enzyme solution (100 μl of 0.4 U/ml collagenase-Type IV from Clostridium histolyticum) and different concentrations of the material in DMSO (80 μl) were preincubated in a microplate for 10 minutes. After preincubation, 20 μl of the substrate DQ gelatin (12.5 μg/ml) was added and the fluorescence intensity was measured after 30 minutes. Enzyme activity with DMSO and controls were also taken. The final concentration of DMSO in the reaction mixture was 3%, which did not show any significant effect on the enzyme activity. Calculations: The percentage inhibition is calculated as follows:

$$\% Inhibition = \frac{( B - B C ) - ( T - C )}{( B - B C )} \times 100$$

B—Fluorescence in the presence of enzyme

BC—Flouroescence in the absence of enzyme activity

T—Fluorescence of enzyme activity in the presence of inhibitor

C—Fluorescence of the inhibitor alone

b) Method for Anti-Elastase Activity

The assays were done using the EnzChek elastase assay kit. The substrate is DQ elastin from soluble bovine neck ligament. DQ elastin is labeled with BODIPY FL dye such that the conjugate″s fluorescence is quenched in the presence of inhibitor. The reduction in fluorescence intensity was measured in a microplate reader/Fluostar Optima (emission at 485 nm and excitation at 520 nm.) Procedure: Aliquots of the enzyme solution (100 μl of 0.5 U/ml of elastase from pig pancreas) and different concentrations of the material in DMSO (50 μl) were preincubated in a microplate for 10 minutes. After preincubation, 50 μl of the substrate DQ elastin (25 μg/ml) ( EnzChek) was added and the fluorescence intensity was measured after 30 minutes. Enzyme activity with DMSO and controls were also taken. The final concentration of DMSO in the reaction mixture was 3%, which did not show any significant effect on the enzyme activity.

Calculations: The percentage inhibition is calculated as follows:

$$\% Inhibition = \frac{( B - B C ) - ( T - C )}{( B - B C )} \times 100$$

B—Fluorescence in the presence of enzyme

BC—Flouroescence in the absence of enzyme activity

T—Fluorescence of enzyme activity in the presence of inhibitor

C—Fluorescence of the inhibitor alone

c) Method for Anti-Hyaluronidase Activity

Ref:-1) Sigma method for the enzymatic assay of hyaluronidase 2) Faizyme Assay procedure Ref No: FGAPO45 hyaluronidase 3) Jwu-Sheng Tung, George.E. Mark and Gregory. F. Hollis. Dept of Cellular and Molecular Biology, Merck Research Laboratories, New Jersey 07065: A Microplate assay for Hyaluronidase inhibitors.

Materials and Methods Hyaluronic acid (from human umbilical cord), hyaluronidase (H 3884 from bovine testis), cetyl pyrinidinium chloride and the other reagents were obtained from Sigma. Hyaluronic acid (HA) was dissolved in 300 mM sodium phosphate buffer pH 5.35. Agarose was dissolved in the same buffer and maintained at 55° C. before use. HA solution was preheated to 55° C. and mixed with agarose to give a final concentration of 0.5 mg/ml of HA and 0.8% of agarose. Warm HA-agarose mixture (100 μl) was dispensed into each well of a microplate and allowed to set. For screening of inhibitors, in another microplate, each well was filled with 100 μl of the HA ase (10 units/test in 10 mM sodium phosphate buffer with 77 mM sodium chloride pH 7.0) and 100 μl of the inhibitor dissolved in DMSO were preincubated at 37° C. for 10 minutes. A final concentration of 1% DMSO was used which did not have any effect on the enzyme activity. Enzyme activity with DMSO and controls were also kept. After preincubation 100 μl from these samples were removed and overlaid onto the pre-set HA/agarose gels wells in triplicates and were incubated at 37° C. for 45 minutes. After incubation enzyme samples were removed, and each well was filled with 100 μl of 10% aqueous cetyl pyridinium chloride. The absorbance was measured in a microplate reader/Fluostar Optima at 600 nm after 10 minutes, at room temperature. A standard plot was also done with the same procedure using different concentrations of hyaluronic acid and a linearity of R²=0.99 was observed between 0.1 to 0.7 mg/ml of HA concentration.

Calculations: The results are expressed as IC 50 values, the concentration at which the compound inhibits half the original hyaluronidase activity. The percentage of inhibition is calculated as follows:

$$\% Inhibition = \frac{( \{ E C - E A ) - ( E C - [ T - T C ] \} \times 100}{( E C - E A )}$$

where

EC—Absorbance in the absence of enzyme and inhibitor

EA—Absorbance in the presence of enzyme activity

T—Absorbance of enzyme activity in the presence of inhibitor

TC—Absorbance of the inhibitor alone

Results: The findings indicate that while Licorice extract containing 90% glabridin has higher collagenase inhibition, Licorice extract containing 40% Glabridin is better as elastase and hyaluronidase Inhibitor

Licorice extract containing 4% Glabridin has a disadvantage of being dark reddish brown colored which invariably colors the product. This can be overcome by use of 40% Glabridin which is pale yellow to off white in color.

In order to formulate an effective cosmetic product, the individual ingredients have to be solublized in cosmetically acceptable solvents. The following table indicates the ease of solubility of glabridin and the licorice extract in contrast to Ursolic acid, a terpenoid compound used in cosmetics for similar claims. The data clearly indicate that glabridin is much more easily soluble than Ursolic acid. This data indicate the ease of the use of glabridin for cosmetic formulations.

While the invention has been described with particular reference to certain embodiments thereof, it should be understood that changes and modifications may be made which are within the skill of the art without affecting the spirit of invention as mentioned under the claims.

1. Compositions containing glabridin or a licorice extract containing a minimum of 4-90% of glabridin, as metalloprotease inhibiting component in cosmetic topical or oral formulations

2. Cosmetic composition incorporating licorice extract containing 4% to 90% glabridin useful in anti-wrinkle and anti-aging products.

3. Composition incorporating licorice extract containing minimum of 4% of Glabridin useful in providing elasticity, firmness, tone and texture to the skin.

4. Composition containing licorice extract with minimum of 4% Glabridin useful in reducing lines and wrinkles associated with normal aging or photoaging.

5. Composition containing licorice extract with minimum of 4% Glabridin useful in preventing skin and hair damage due to UV rays.

6. Composition containing licorice extract with minimum of 4% Glabridin useful in inflammation, itch, prickly heat

7. Composition containing licorice extract with minimum of 4% Glabridin useful in diaper rashes in baby products, as topical massage or toning oils or emulsion for babies.

8. Composition containing licorice extract with minimum of 4% Glabridin useful orally, in maintaining and alleviating conditions of arthritis, joint immobility, osteoarthritis and conditions manifested due to increased activity of elastase, collagenase and hyaluronidase enzymes.

9. Composition containing licorice extract with minimum of 4% Glabridin can be used in concentrations of 0.01-10%, preferably in concentrations of 0.1-2% depending on the purity of the extract.

10. Composition containing licorice extract with minimum of 4% Glabridin for use alone or in combination with other plant extract or chemicals or minerals or natural products, including but not limited to solutions, lotions, creams, powders, drops, sprays as w/o or o/w emulsions, tablets, capsules, soft gelatins, spansules, effervescent preparations among others.
